(19) United States
(12) Patent Application Publication
(10) Pub. No.: US 2010/0262035 A1
(43) Pub. Date: Oct. 14, 2010

(54) Portable Sleep Apnea Monitor

(75) Inventor: Narayanan M. Subramanian, Marietta, GA (US)

Correspondence Address: NARAYANAN M. SUBRAMANIAN, 4251 CREEK HAVEN DR., MARIETTA, GA, 30062, US

(73) Assignee: INVENTION DYNAMICS, LLC, Marietta, GA (US)

(21) Appl. No.: 12/420,855

(22) Filed: Apr. 9, 2009

(51) Int. Cl. A61B 5/08

(52) U.S. Cl. 600/534

(57) ABSTRACT

A belt made of a substantially non-elastic, flexible material with a buckle assembly having a sensor to sense breathing by means of the movement of the abdomen, an evaluator to use the information from the sensor to determine the occurrence of an apnea event, a vibrator motor to stimulate the wearer when an apnea event occurs, an alarm unit to alert people around the wearer should the wearer not respond to the stimuli from the vibrator motor, a rechargeable battery to power the electrical/electronics units and a data transfer port to transfer data from the evaluator to a display console. The display console is a stand-alone unit that receives data from the evaluator and displays the time of each apnea event that occurred.

### REFERENCE CITED

U.S. Pat. No. 3,480,010—Nov. 25, 1969—Crossley
U.S. Pat. No. 3,696,377—Oct. 3, 1972—Wall
U.S. Pat. No. 3,782,368—Jan. 1, 1974—Reibold
U.S. Pat. No. 4,494,553—Jan. 22, 1985—Sciarra et al.
U.S. Pat. No. 4,593,686—Jun. 10, 1986—Lloyd et al.
U.S. Pat. No. 4,715,367—Dec. 29, 1987—Crossley
U.S. Pat. No. 4,889,131—Dec. 26, 1989—Salem, Robert J et al.
U.S. Pat. No. 5,191,893—Mar. 9, 1993—Reiten
U.S. Pat. No. 5,265,624—Nov. 30, 1993—Bowman
U.S. Pat. No. 5,295,490—Mar. 22, 1994—Dodakian, Wayne S.
U.S. Pat. No. 5,357,981—Oct. 25, 1994—Eilam et al.
U.S. Pat. No. 5,454,376—Oct. 3, 1995—Stephens et al.
U.S. Pat. No. 5,666,104—Sep. 9, 1997—Pollack et al.
U.S. Pat. No. 5,902,250—May 11, 1999—Verrier et al.
U.S. Pat. No. 6,162,183—Dec. 19, 2000—Hoover
U.S. Pat. No. 6,267,730—Jul. 31, 2001—Pacumas
U.S. Pat. No. 6,357,444—Mar. 19, 2002—Parker
U.S. Pat. No. 6,360,615—Mar. 26, 2002—Smela
U.S. Pat. No. 6,384,728—May 7, 2002—Kanor et al.
U.S. Pat. No. 6,666,830—Dec. 23, 2003—Lehrman et al.
U.S. Pat. No. 6,935,335—Aug. 30, 2005—Lehrman et al.
2002/0,032,386—Mar. 14, 2002—Sackner et al.
2002/0,097,155—Jul. 26, 2002—Cassel et al.
2006/0,206,035—Sep. 14, 2006—Friedrichs et al.
2006/0,258,916—Nov. 16, 2006—Pietersen
2007/0,208,232—Sep. 6, 2007—Kovacs
2007/0,256,695—Nov. 8, 2007—Crocetti
2007/0,293,781—Dec. 20, 2007—Sims et al.
2008/0,108,903—May 8, 2008—Ben-Oved et al.
2008/0,109,047—May 8, 2008—Pless
2008/0,119,896—May 22, 2008—Wong et al.
2008/0,183,095—Jul. 31, 2008—Austin et al
2008/0,264,426—Oct. 30, 2008—Walker, James
2008/0,269,583—Oct. 30, 2008—Reisfeld, Daniel
2008/0,269,832—Oct. 30, 2008—Wong et al.
2008/0,275,356—Nov. 6, 2008—Stasz et al.
2008/0,297,357—Dec. 4, 2008—Golbin et al.

### FIELD OF INVENTION

The present invention relates generally to monitoring sleep apnea. The present invention relates more particularly to a compact, wearable unit that can recognize the occurrence of sleep apnea event and urge the wearer to start breathing again.

### BACKGROUND OF INVENTION

Sleep apnea is the condition where people stop breathing for extended periods of time while sleeping. Generally, after a prolonged period of not breathing, the brain sends a stimulus and the person starts breathing again. A person can have multiple instances of sleep apnea during the course of a night's sleep. Several patents have been issued in this area. Some inventions relate to jackets with bumps on the back to prevent the wearer from lying on his/her back. In others, sensors are attached to the patient and the condition monitored in a hospital. This is a diagnostic method which does not help the sufferer in a home setting. In a third variety of solution, air is pumped into the nostril through a face mask. This is not a preferred method since it is inconvenient for the patient and interferes with normal family life.

A few patents are either pending or have been granted where the unit is small enough for the patient to wear it daily at night without it interfering with the normal life of the wearer. U.S. Pat. No. 6,162,183 and application number 2008/0269583 use optical emitter/detector units. In this, either the emitter or the detector moves with the movement of the abdomen during breathing. The constant movement of the emitter or detector makes the unit unreliable. The unit can easily get out of alignment after a brief period of use and so become useless. U.S. Pat. No. 4,494,553 and application number 2008/0183095 use inductance sensors to monitor breathing. In these units, the inductance of a coil is varied by a moving rod that moves with breathing. As paragraph 59 of application 2008/0183095 points out, it uses a “complex programmable logic device 516”. U.S. Pat. No. 5,454,376 and application 2008/0119896 use an elastic fabric with strain gauge. Because of the elasticity of the body, depending on the pressure applied by the belt, the abdominal movement can be pronounced around the belt and minimal under the belt. When the belt is elastic, the stretching of the fabric is distributed around the circumference of the belt and so the detection unit has to be very sensitive. Too sensitive an instrument not only costs more, it can give false readings because of extraneous “noise”. The strain gauges also increase the cost of the unit. U.S. Pat. Nos. 4,889,131, 4,909,260 and 6,267,730 and application 2008/0275356 use a piezo electric sensor on an elastic belt, causing the same problems mentioned above.

All the units that use an elastic belt to mount the sensor have another common drawback. In daily use, the elasticity of the belt can weaken and become useless.

Application 2006/0258916 uses a pressure transducer to monitor breathing. Since the initial pressure itself can vary every time the belt is worn, finding acceptable pressure variation to activate the alarms will be complicated. Also the cost of the pressure transducer is relatively high.

In U.S. Pat. No. 5,295,490, the unit has a spring going all around the belt that is worn. This will be irritating to the wearer. Also, it uses a lot of mechanical parts. Although the inventor specifies material to be used to reduce friction, there will still be considerable friction that the intended result will not be achieved in reality. The increase in the number of parts also increases the cost of manufacture.

Application 2008/0108903 uses a spring loaded roller that keeps the belt coiled. The unit is complicated and not reliable. Since the belt extends out equally from the casing, the casing will be on the back of the wearer. When a person lies on his back, the casing will cause discomfort. Also, while lying on the back, the weight of the body will pin the belt to the mattress and prevent the belt from coiling and uncoiling. This will set off false alarm that the person has stopped breathing. The signal generators are also not of the simple type, thus adding to the cost and complexity.

### SUMMARY OF INVENTION

The primary objective of the present invention is to come up with a sleep apnea monitor that is light in weight, comfortable to wear at night and does not come in the way of normal family life.

Another objective of the present invention is to increase the reliability of the unit by reducing the number of moving parts.

A third objective of the present invention is to bring down the cost of manufacture through the use of minimal as well as less expensive parts so that the unit is affordable for the vast majority of patients.

The foregoing objectives are attained by having a substantially non-elastic belt with a buckle having a small spring actuated contact and a microcontroller to register the contact output and determine if an apnea event has occurred.

In this respect, before explaining at least one embodiment of the invention in detail, it is to be understood that the invention is not limited in its application to the details of construction and to the arrangements of the components set forth in the following description or illustrated in the drawing(s). The invention is capable of other embodiments and of being practiced and carried out in various ways. Also, it is to be understood that the phraseology and terminology employed herein are for the purpose of description and should not be regarded as limiting.

As such, those skilled in the art will appreciate that the concept, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

### BRIEF DESCRIPTION OF THE DRAWINGS

FIG. 1 is the front view of a preferred embodiment of the sleep apnea detector. It consists of a substantially non-elastic belt with a buckle assembly. The buckle assembly contains a buckle, a sensor, an evaluator, a vibrator motor, an alarm unit and a rechargeable battery to power the sensor, evaluator, vibrator motor and alarm unit.

FIG. 2 and FIG. 3 show the construction of the buckle assembly of the preferred embodiment of the sleep apnea detector with the internal parts revealed.

FIG. 4 is the exploded view of an electrically conductive contact plate—spring—slot sub-assembly.

FIG. 5 is the bottom view of the buckle assembly of the preferred embodiment of the sleep apnea detector showing a connector for data transfer and a switch to enable and disable sleep apnea determination by the evaluator.

FIG. 6 shows the charger/apnea event display console of the preferred embodiment of the sleep apnea detector. This unit is used to charge the rechargeable battery in the buckle assembly as well as display the time of each apnea event that occurred since the previous display.

FIG. 7 shows the apnea event decision logic used to determine an apnea event.

FIG. 8 is the schematic diagram of the belt-sensor assembly—display console interaction.

FIG. 9 show the construction of the buckle assembly for a second preferred embodiment of the invention with the internal parts revealed.

FIG. 10 is the bottom view of the buckle assembly for the second preferred embodiment of the invention, showing a power input socket for charging the rechargeable battery and a switch to enable and disable sleep apnea determination by the evaluator.

FIG. 11 shows the apnea event display console for the second preferred embodiment of the invention. The unit is used to display the time of each apnea event that occurred since the previous display.

### DETAILED DESCRIPTION OF THE INVENTION

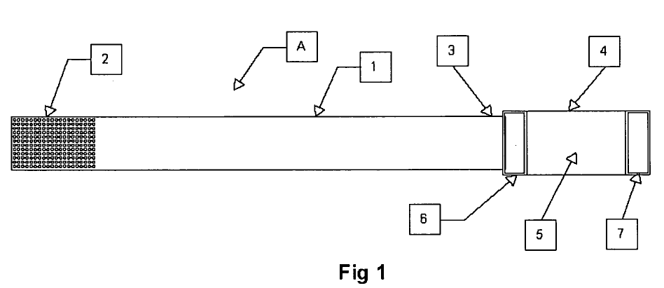

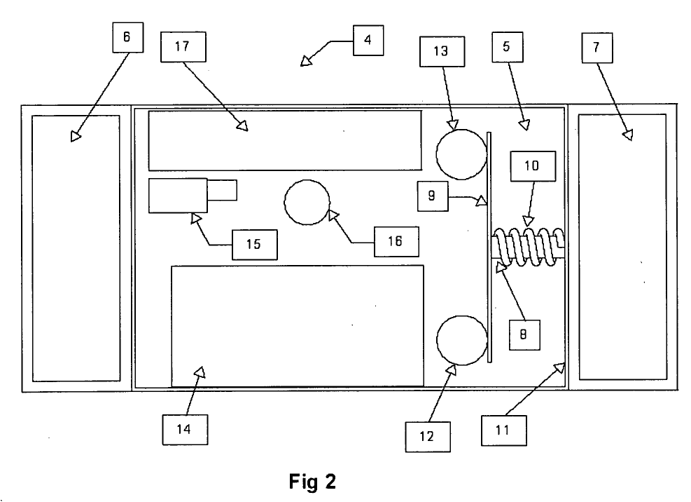

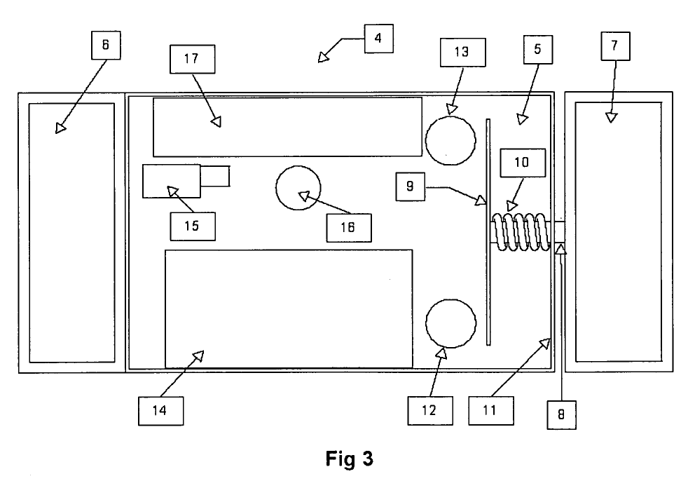

FIG. 1 refers to a preferred embodiment of the present invention. Here, a belt-sensor unit, generally referred to as A, has a belt 1, attached to a buckle assembly 4. The buckle assembly has a buckle 5, inside which are located a sensor, an evaluator, a vibrator, an alarm unit and a rechargeable battery. These will be explained further while describing FIG. 2 and FIG. 3. On one side of buckle 5, is a slot 6, which is fixedly attached to the buckle. On the other side of buckle 5 is another slot 7, which is movably attached to the buckle. Slot 6 and slot 7 can be of any shape—circular, square, rectangular etc. as long as the belt means can pass through the opening in slot 6 and slot 7.

Belt 1 is made from a substantially non-elastic, flexible material. One end of belt 1 that is distal to the buckle assembly has a hook and loop fastener. This end will henceforth be referred to as the ‘distal end’. The hook and loop fastener, more commonly known as ‘Velcro’ is numbered 2. The other end of the belt, numbered 3, that is proximal to the buckle assembly, may be attached securely to the buckle via slot 6 by any of the known fastening means such as a hook and loop fastener, looping the belt through slot 6 and binding end 3 with the belt body 1 by means of rivets, glue, stitching etc. This end will henceforth be referred to as the ‘proximal end’.

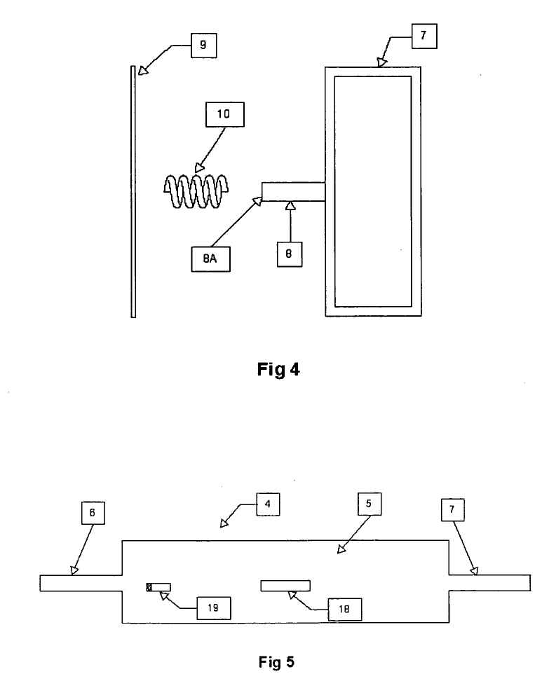

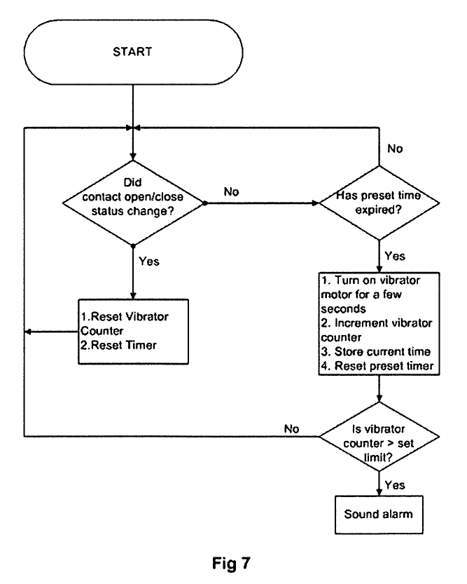

FIG. 2 and FIG. 3 show the buckle assembly 4 with its internal parts revealed. FIG. 4 shows the assembly of spring 1 0, electrically conductive contact plate 9 and slot 7. As explained in FIG. 1, slot 7 is movably attached to buckle 5. Shaft 8 is fixedly attached to slot 7. Shaft 8 passes through a hole in wall 11 of buckle 5. A compression spring 10 passes loosely over shaft 8. Electrically conductive contact plate 9 is attached to the free end, 8A, of shaft 8 such that compression spring 10 is compressed between contact plate 9 and wall 11 when slot 7 is pulled out. The hole in wall 11 is big enough for the shaft to slide smoothly in and out but small enough to prevent the compression spring from come out through the hole. Contact pins 12 and 13 are placed such that compression spring 10 will push contact plate 9 to touch them both when slot 7 is not pulled out. This situation is illustrated in FIG. 2. When slot 7 is pulled out, the contact between pins 12 and 13 via contact plate 9 is broken. This situation is illustrated in FIG. 3. Contact pins 12, 13 and contact plate 9 together act as a switch. The buckle assembly also houses an evaluator, 14, that receives the on/off signal from the switch formed by contact plate 9 and contact pins 12 and 13. The time interval between the switch making the contact, also called an ‘On’ signal, and a contact break, also called an ‘Off’ signal, provides the information needed to determine the occurrence of sleep apnea. The evaluator, which can be a microcontroller, has a timer with a preset time value. This time value is in excess of the normal breathing time period. If the time interval between an ‘On’ signal and an ‘Off’ signal is greater than this preset time interval, then the evaluator determines it to be a sleep apnea event. The sleep apnea determination logic is illustrated in FIG. 7. The buckle assembly also houses a vibrator motor, 15, an alarm, 16 and a rechargeable battery 17. The vibrator motor and the alarm are controlled by the evaluator. When the evaluator determines that an apnea event has occurred, it energizes the vibrator motor. It increments the vibrator counter, resets the preset timer and stores the current time in memory as the apnea event time. If the patient does not respond by breathing even after a few instances of stimulation by the vibrator motor, the evaluator energizes the alarm. The rechargeable battery powers all these electrical/electronics instruments.

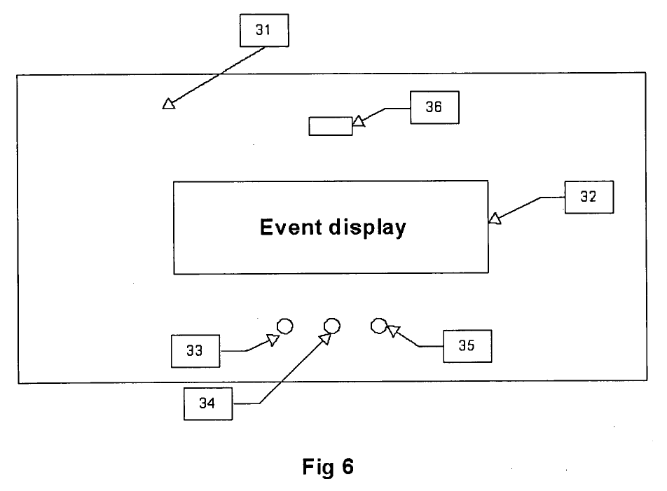

FIG. 5 shows the bottom view of the buckle assembly. It has a switch 19 to enable and disable the monitoring of sleep apnea events by the evaluator, and a data transfer port 18, to communicate with a stand-alone display console 31 shown in FIG. 6. In this preferred embodiment of the invention, port 18 is used to exchange apnea event information with display console 31 and also obtain power from the display console to recharge battery 17.

FIG. 6 shows the display console 31. It has a display unit 32 to display the current time or the time of each apnea event. The display console also has a complimentarily configured data transfer port 36, which removably mates with data transfer port 18 on the buckle assembly. Three buttons, numbered 33, 34 and 35 are available for the user to control the display. By pressing the proper sequence of buttons, the user can set the time displayed; transfer the time information to the buckle assembly using data transfer port 36 or scroll through the apnea events information acquired from the buckle assembly.

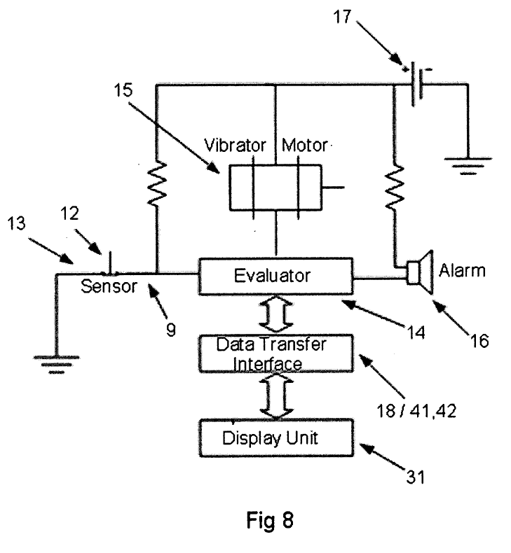

Using switch 19, the user first disables the monitoring of sleep apnea occurrences by the evaluator. Then he plugs in the display console 31 into the electrical outlet. He then plugs the belt-sensor unit into the display console using data transfer ports 18 and 36. At this time, the rechargeable battery in the buckle assembly starts getting charged with power from the display console. If necessary, using the appropriate sequence of buttons 33, 34 and 35, the user corrects the time stored on the evaluator and displayed on the display console 31. Similarly, using buttons 33, 34 and 35, the user displays on display console 31, the time of each apnea event stored in the memory of the evaluator. FIG. 8 shows the schematic diagram of the interaction between the sensor and display console.

When the user goes to bed, he removes the belt-sensor unit from the display console and wears it around the abdomen. The belt-sensor unit is worn with just sufficient tightness such that the wearer is comfortable while at the same time, the movable slot 7 is pulled out against the compression force of the spring when the wearer inhales and the abdomen bulges. After fastening the belt-sensor unit properly, the wearer enables the sleep apnea determination function of the evaluator using switch 19. During inhalation, the abdomen bulges, pulling slot 7 out. This breaks the electrical contact between contact pins 12 and 13 by pulling contact plate 9 away from pins 12 and 13. When the wearer exhales, the abdomen moves in, and the spring force pulls slot 7 back. This causes the contact plate to electrically connect contact pins 12 and 13. During breathing, each time the electrical contact between pins 12 and 13 is made or broken, the evaluator unit 14 receives the signal and acknowledges the event by resetting the preset timer and the vibrator counter. If the preset time value is exceeded before the electrical contact is made or broken, the evaluator determines it to be an apnea event and energizes the vibrator motor for a few seconds. It increments the vibrator counter, resets the timer and stores the current time in memory as the apnea event time. The vibration from the vibrator motor stimulates the wearer to start breathing. When the timer expires again, if the wearer has not responded by breathing again, the evaluator energizes the vibrator motor once more. This process is repeated a preset number of times. If the wearer still has not responded to the stimuli, the alarm unit is energized to sound an alarm to notify anyone present in the vicinity to take appropriate medical action.

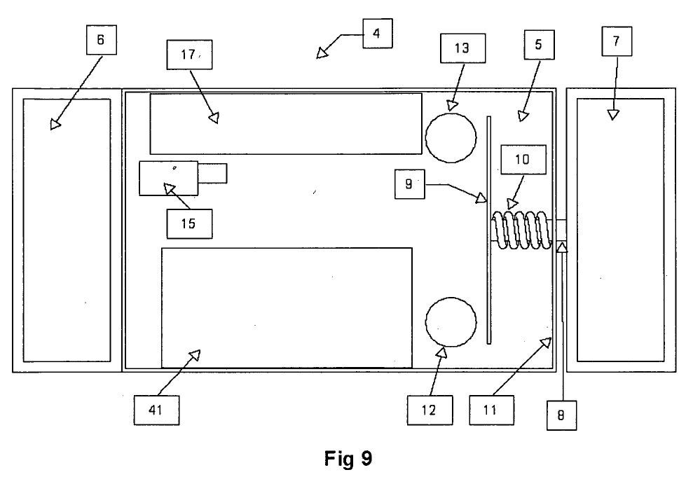

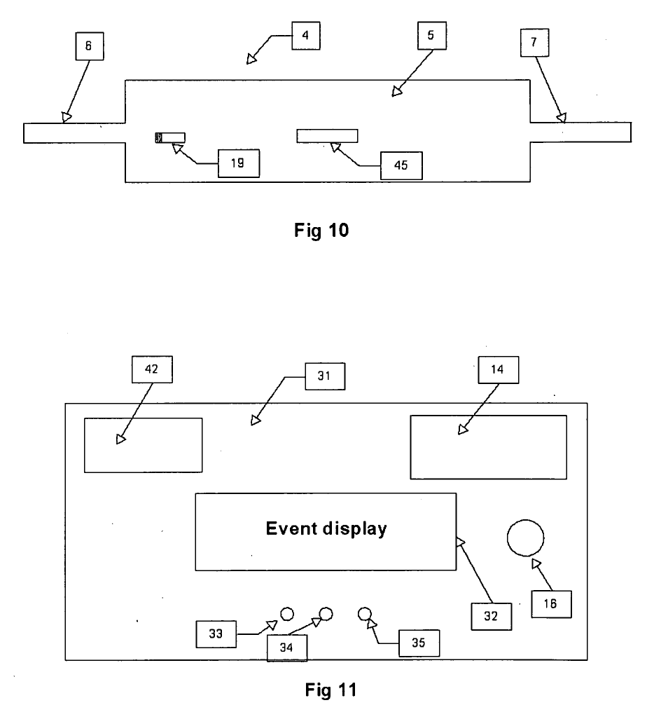

FIG. 9, FIG. 10 and FIG. 11 refer to a second preferred embodiment of the present invention. In this second preferred embodiment, the alarm unit 16 and the evaluator 14 are placed in the display console, 31. The buckle has a transmitter/receiver unit, 41, which is powered by the rechargeable battery, 17. The display console, 31, also has a transmitter/receiver unit, 42. Unit 41 and unit 42 work seamlessly to exchange data between them. When the contact between contact pins 12 and 13 is made or broken by the contact plate 9, unit 41 transmits the information wirelessly to unit 42 in the display console. When the evaluator, using unit 42, transmits the command to start or stop the vibrator motor, unit 41 receives the command and operates the vibrator motor. Similarly, data port 18 has been replaced by a power input socket 45. The rechargeable battery, 17 is connected to the power input socket 45. The user can recharge the rechargeable battery by connecting a commercially available charger between the power input socket 45, and the electrical outlet. The rechargeable battery powers the transmitter/receiver unit 41 as well as the vibrator motor. The data transfer port, 36, of the first preferred embodiment is not needed in this second embodiment since data transfer takes place using the transmitter/receiver units on the buckle assembly and the display console.

The user first deactivates switch 19. This information is transmitted by unit 41 to unit 42. Unit 42 passes this signal to the evaluator to stop monitoring the occurrences of sleep apnea events. He then charges the battery by connecting the buckle assembly to the charger unit using power input socket 45. Then he plugs in the display console to the electrical outlet. Using the proper sequence of buttons 33, 34 and 35, the user sets the current time of display on display console 31.

When the user goes to bed, he removes the belt-sensor unit from the charger and wears the unit around the abdomen. The belt-sensor unit is worn with just sufficient tightness such that the wearer is comfortable while at the same time, the movable slot 7 is pulled out against the compression force of the spring when the wearer inhales and the abdomen bulges. After fastening the belt-sensor unit properly, the wearer activates switch 19. This information is transmitted by unit 41 to unit 42. Unit 42 passes this signal to the evaluator to start determination of sleep apnea events. During inhalation, the abdomen bulges, pulling slot 7 out. This breaks the electrical contact between contact pins 12 and 13 by pulling contact plate 9 away from pins 12 and 13. When the wearer exhales, the abdomen moves in, and the spring force pulls slot 7 back. This causes the contact plate to electrically connect contact pins 12 and 13. During breathing, each time the electrical contact between pins 12 and 13 is made or broken, the transmitter/receiver unit 41 sends a signal to the transmitter/receiver unit 42 on the display console. Unit 42 passes on the signal to evaluator 14. On receipt of the signal, evaluator 14 resets the preset timer and the vibrator counter. If the preset time value is exceeded before the signal is received by the evaluator, the evaluator determines it to be an apnea event and sends a signal through unit 42 to unit 41 to energize the vibrator motor for a few seconds. It increments the vibrator counter, resets the preset timer and stores the current time in memory as the apnea event time. The vibration from the vibrator motor stimulates the wearer to start breathing. If the wearer has not responded by breathing again, there will not be any signal arriving from unit to unit 42. When the timer expires again, if there is no signal from unit 41, the evaluator sends a signal through unit 42 to unit 41 to energize the vibrator motor once more. This process is repeated a preset number of times. If the wearer still does not respond to the stimuli, the alarm unit is energized to sound an alarm to notify anyone present in the vicinity to take appropriate medical action.

In the first preferred embodiment as well as the second preferred embodiment, the proximal end of belt 1 can be fixedly attached to moveable slot 7 instead of fixed slot 6 and the distal end of the belt can be fastened using slot 6 instead of slot 7. This does not change the working of the unit.

Since the belt material is substantially non-elastic, the full force from the abdominal expansion during inhalation is used to compress the spring. Since there is only one moving part, friction is greatly reduced. The sensor being a simple but efficient contact switch, the cost of the unit is substantially brought down.

What is claimed is:

1. A portable sleep apnea monitoring system comprising:
a belt comprising a substantially non-elastic, flexible material and having a distal end and a proximal end, the belt configured for secure placement generally encircling the abdomen and respiratory areas of a patient;
a buckle assembly having a buckle and a first slot configured to receive said proximal end of said belt and a second slot configured to receive said distal end of said belt, the buckle assembly configured to expand and contract in response to respiration of the patient;
a sensor assembly, disposed within said buckle and configured to detect repeatedly a plurality of expansions and contractions of said buckle assembly;
an evaluator, disposed within said buckle, configured to receive a plurality of expansions and contractions signals from said sensor assembly, and configured to make calculations based upon a plurality of time intervals collected from the plurality of expansions and contractions of said buckle assembly, and thereby detect each sleep apnea event, and store the time of each sleep apnea event detected for later upload to an external device;
a vibrator motor, disposed within said buckle and configured to be actuated by said evaluator when said evaluator determines that a sleep apnea event has occurred and thereby to inform the patient by vibration;
an alarm, disposed within said buckle and configured to be actuated by said evaluator when said evaluator determines that the patient is unresponsive to said vibrator motor actuations; and
a rechargeable battery, disposed within said buckle and configured to provide rechargeable power to the portable sleep apnea monitor.

2. The portable sleep apnea monitoring system of claim 1, wherein said evaluator is a microcontroller.

3. The portable sleep apnea monitoring system of claim 1, wherein said sensor assembly further comprises a switch formed by at least one contact pin, a contact plate to make and break contact with the at least one contact pin, and at least one compression spring placed around a shaft coupled to said second slot of said buckle assembly on a first end and coupled to said contact plate on a second end, and configured to separate said contact plate from the at least one contact pin upon patient inhalations.

4. The portable sleep apnea monitoring system of claim 1, further comprising:
a switch disposed upon said buckle assembly and configured to enable and disable monitoring of the occurrences of sleep apnea events by said evaluator.

5. The portable sleep apnea monitoring system of claim 1, further comprising:
a data transfer port disposed upon said buckle assembly, configured to provide transfer of data to an external device, and configured to receive power from the external device.

6. The portable sleep apnea monitoring system of claim 1, further comprising:
a display console configured for data transfer connectivity to said evaluator.

7. The portable sleep apnea monitoring system of claim 6, further comprising:
A display unit to display a plurality of stored data from said evaluator, including a time of each sleep apnea event determined by said evaluator; and
a plurality of control buttons disposed upon said display console and operable to configure what is displayed on said display unit.

8. The portable sleep apnea monitoring system of claim 6, wherein said display console is an external device.

9. The portable sleep apnea monitoring system of claim 8, wherein said display console is a personal computer system.

10. The portable sleep apnea monitoring system of claim 8, wherein said display console is not a personal computer system.

11. A portable sleep apnea monitoring system comprising:
a belt comprising a substantially non-elastic, flexible material and having a distal end and a proximal end, the belt configured for secure placement generally encircling the abdomen and respiratory areas of a patient;
a buckle assembly having a buckle and a first slot configured to receive said proximal end of said belt and a second slot configured to receive said distal end of said belt, the buckle assembly configured to expand and contract in response to respiration of the patient;
a sensor assembly, disposed within said buckle and configured to detect repeatedly a plurality of expansions and contractions of said buckle assembly;
a first transmitter/receiver disposed within said buckle and configured to transmit a plurality of data concerning the plurality of expansions and contractions of said buckle assembly to an external device; and
a vibrator motor, disposed within said buckle and configured to be actuated by an external device when said external device determines that a sleep apnea event has occurred and thereby to inform the patient by vibration.

12. The portable sleep apnea monitoring system of claim 11, further comprising:
a rechargeable battery, disposed within said buckle and configured to provide rechargeable power to the portable sleep apnea monitor; and
a power input socket configured for connectivity to a charger unit to charge said rechargeable battery.

13. The portable sleep apnea monitoring system of claim 11, wherein said sensor assembly further comprises a switch formed by at least one contact pin, a contact plate to make and break contact with the at least one contact pin, and at least one compression spring placed around a shaft coupled to said second slot of said buckle assembly on a first end and coupled to said contact plate on a second end, and configured to separate said contact plate from the at least one contact pin upon patient inhalations.

14. The portable sleep apnea monitoring system of claim 11, further comprising:
a display console configured to receive, from said first transmitter/receiver, the plurality of data concerning the plurality of expansions and contractions of said buckle assembly;
the display console having a second transmitter/receiver configured to communicate with said first transmitter/receiver disposed within said buckle assembly;
the display console having an evaluator configured to receive a plurality of expansions and contractions signals from said second transmitter/receiver and configured to make calculations based upon the plurality of time intervals collected from the plurality of expansions and contractions of said buckle assembly, and thereby detect each sleep apnea event, and store the time of each sleep apnea event detected for later display;
the display console having an alarm configured to be actuated by said evaluator when said evaluator determines that the user is unresponsive to said vibrator motor actuations; and
the display console having a display unit configured to display a plurality of stored data from said evaluator, including a time of each sleep apnea event determined by said evaluator.

15. The portable sleep apnea monitoring system of claim 14, wherein said second transmitter/receiver is configured to communicate with said first transmitter/receiver that said vibrator is to be actuated.

16. The portable sleep apnea monitoring system of claim 14, further comprising:
a plurality of control buttons disposed upon said display console and operable to configure what is displayed on said display unit.

17. The portable sleep apnea monitoring system of claim 14, wherein said evaluator is a microcontroller.

18. The portable sleep apnea monitoring system of claim 14, wherein said display console is an external device.

19. The portable sleep apnea monitoring system of claim 18, wherein said display console is a personal computer system.

20. The portable sleep apnea monitoring system of claim 18, wherein said display console is not a personal computer system.

21. The portable sleep apnea monitoring system of claim 11, further comprising:
a switch disposed upon said buckle assembly and configured to enable and disable monitoring of the occurrences of sleep apnea events by said evaluator.
